(19) United States
(12) Patent Application Publication
(10) Pub. No.: US 2015/0366777 A1
(43) Pub. Date: Dec. 24, 2015

(54) COMBINATION OF SKIN PROTECTION AGAINST DAMAGES CAUSED BY INFRARED RADIATION

(71) Applicant: Hypermarcas S.A., Sao Paulo (BR)

(72) Inventors: Liliana Bechelli de Oliveira TORLONI, Sao Paulo (BR); Andréia Feital da Costa Pereira, Barueri (BR)

(21) Appl. No.: 14/737,862

(22) Filed: Jun. 12, 2015

(30) Foreign Application Priority Data
Jun. 18, 2014 (BR) 10 2014 014999 6

(51) Int. Cl. A61K 8/64; A61Q 17/04; A61K 8/97; A61K 8/60; A61K 8/06

(52) U.S. Cl. CPC A61K 8/645; A61K 8/606; A61K 8/06; A61K 8/97; A61Q 17/04; A61K 2800/78

(57) ABSTRACT

A combination of at least one SIRT1 peptide activator and at least one guanosine derivative alarmone, in a dermatological cosmetic for providing protection against damages caused by infrared radiation.

The present invention relates to a combination of chemical compounds, aimed mainly to use in the dermatological cosmetic field, which providing protection against damages caused by infrared radiation. More particularly, the invention relates to a combination comprising a SIRT1 peptide activator and a guanosine derivative alarmone.

### BACKGROUND OF THE INVENTION

In the text below, “IR” means infrared radiation, of wavelength between 700 and 1500 nm, “UVA” means ultraviolet A-radiation, between 320 and 400 nm, and “UVB” means ultraviolet B-radiation, between 290 and 320 nm.

Excessive exposure to solar radiation causes adverse effects to health, from premature skin aging, which becomes prematurely blemished, less flexible and wrinkled, to the appearance of certain types of skin cancer.

While the need to protect human skin against UVA and UVB has been receiving attention for at least 30 years, less attention has been given to IR effects which, until recently, was merely mentioned as a generator for heat sensation, causing erythemas ab igne on the skin (e.g., sunburn). It was believed until recently that, as a low-energy radiation, its inability to trigger photochemical reactions would result in low contribution to cutaneous carcinogenesis. However, there is an increasing number of evidences showing that IR not only contributes to premature skin aging, amplifying the deleterious effects of UV, changing the vasculature (telangiectasies), producing free radicals, modifying the histone binding properties and impairing the DNA repair processes.

For protection against UVA and UVB, the current technology of sun protection products is improving, including ingredients in topical formulations that reflect or absorb such radiations. These ingredients are chemical (for example, avobenzone and benzophenone) or physical (for example, metal oxides such as zinc and titanium). Some of them provide protection against both UVA and UVB. However, most of those ingredients do not provide protection against IR, which penetrate deeper into the skin than UVA or UVB and, as they are absorbed, spreads heat by conduction and convection.

A few proposals for compositions that provide protection against IR are found in the prior art:

Document U.S. Pat. No. 5,876,699 discloses that titanium dioxide provides absorption and scattering of IR radiation, but the amount that can be used in a composition must be less than 4%, to avoid bleaching effect;

Document U.S. Pat. No. 4,822,600 discloses that fumed silica, comprising between 1 and 10% of a composition, preferably in combination with UV blockers, provide protection against IR;

Document U.S. 5,427,771 discloses that a cosmetic composition comprising titanium dioxide flakes with particle size between 1.5 and 25 μm, and an adjuvant chosen among water, lower monohydric alcohols and C1-C6 polyols.

Document JP2006151917 discloses a cosmetic composition comprising a mixture of titanium dioxide with particle size between 0.5 and 5 μm, and a boron nitride powder.

Skin protection against IR is a knowledge area under development, in continuous search for alternatives which provide efficiency and compatibility with topical compositions currently used and which already provide protection against UVA and UVB.

### DESCRIPTION OF THE ATTACHED FIGURES

FIG. 1—Effect of combination of the invention on viability of human dermal fibroblasts after 48 h, assessed by MTT test—chart of one-way analysis of variance of Kruskal-Walis test—Dunnett method—mean±standard error, *p<0.05 vs. untreated.

FIG. 2—Effect of combination of the invention on MMP1 expression induced by IR-A in human dermal fibroblasts—chart of one-way analysis of variance by ranks—Kruskal-Walis test—Student-Newman-Keuls method; *p<0.05 vs. untreated (0% combination of the invention, without IR-A); +p0.05 vs. IR-A, isolated; #p<0.05 vs treatment indicated.

### BRIEF DESCRIPTION OF THE INVENTION

The present invention relates to a combination of active principles for use in the dermatological cosmetic field, which provides protection against damages caused by infrared radiation (IR).

More particularly, the invention relates to a combination for skin protection against damages caused by infrared radiation, characterized by comprising a SIRT1 peptide activator (ingredient A) and a guanosine derivative alarmone (ingredient B).

### Ingredient A

Sirtuin enzymes (SIRT) are a highly conservative family of deacetylase proteins that depend on NAD+ (nicotinamide adenine dinucleotide) for its activity, with important participation in histone deacetylation. 7 sirtuin are known in mammals, and such proteins have been linked to caloric restriction and aging via modulation of energy metabolism, genomic stability and resistance to stress.

The different sirtuins of mammals have different subcellular locations, and SIRT1 usually has a nuclear location, although there are mentions to cytoplasmic SIRT1, associated to apoptosis, differentiation and oncogenic transformation.

There are indications in the prior art that SIRT proteins, in particular SIRT1, are expressed in skin cells, and such expression relates to different stresses that skin cells suffer from. The induction of expression of such proteins would, therefore, improve cell protection and aid in the reaction against environmental aggressions and aging.

It is known that peptides are SIRT1 activators. According to the sense employed herein, among the compounds of peptide nature capable of activating SIRT1, there are fragments of proteins, peptide and polypeptide fragments and all sequences of two or more amino acids linked by peptide bonds. Peptide fragments obtained from hydrolyzed rice extracts are adequate, for example, as mentioned in patent U.S. Pat. No. 8,703,161, particularly the fragment mentioned as SEQ ID No. 8, which is commercially available under the name Orsirtine® GL, commercialized by the ISP Vincience company (Ashland group): G-L-Y-D-N-L-E Gly-Leu-Tyr-Asp-Asn-Leu-Glu

### Ingredient B

Alarmones are hormonal intracellular signal molecules produced in response to adverse environmental conditions, such as oxidative stress. In some contexts, they are associated with the protection and repair of mitochondrial DNA.

Adequate to the invention are the alarmones derived from guanosine, more particularly those chosen among guanosine monophosphate (RN: 85-32-5), guanosine diphosphate (RN: 146-91-8), guanosine triphosphate (RN: 86-01-1), 5′-diphosphate-3′-guanosine monophosphate (RN: 58902-76-4), guanosine tetraphosphate (RN: 33503-72-9), diguanosine tetraphosphate (RN: 4130-19-2) and guanosine pentaphosphate (RN:-38918-96-6). In a particular embodiment, diguanosine tetraphosphate is used, for example as commercially supplied by ISP Vincience company (Ashland group), under the name GP4G® (extract of Artemia saline).

Adequately and in a non-exclusive manner, the combination of the invention, of protection against damages caused by IR radiation, comprises an ingredient A—which is, at least, one SIRT1 peptide activator, and an ingredient B—at least one guanosine derivative alarmone.

The combination of the invention can be used in any dermatological, cosmetic or pharmaceutical topic product, for example, sunscreen, face cream, body cream or any other profiting from protection against the damages caused by IR radiation. Adequately, without excluding any other alternative, the content of the combination in relation to the total weight of the composition which contains it, varies between 0.01% and 2%.

When ingredient A is Orsirtine® and ingredient B is GP4G®, the appropriate ratios in relation to the total weight of the composition which comprises them, without excluding any other, varies between 1:4 and 4:1, particularly between 1.5:1 and 1:1.5.

So, the inventive combination, without excluding any other alternative, comprises ratios between ingredients A and B from 1:10 to 10:1.

The combination of the invention can be formulated with all types of vehicles, for example, oil-in-water, water-in-oil, silicone-in-water, water-in-silicone, water-in-oil-in-water, oil-in-water-in-oil emulsions, etc., cream, lotion, solution, anhydrous bases (lipsticks, powders), gel, paste, ointment, milk, liquid, aerosol, jelly, solid, etc.

The combination of the invention can be formulated with cosmetic and pharmaceutically acceptable ingredients, for example, fragrances, colorants, pigments, adsorbents, emulsifiers, stabilizers, lubricants, solvents, emollients, humectants, film formers, occlusion agents, physical or chemical UV absorbers, essential oils, vitamins, trace metals, anti-irritants, botanic extracts, antimicrobials, antioxidants, chelating agents, pH adjusters, absorbents, exfoliating agents, skin conditioners, thickeners, silicone containing agents, pharmaceutical ingredients and any other cosmetic and pharmaceutically acceptable substrates.

In another aspect, the invention relates to the use of the combination described above in the prevention and repair of damages caused to the skin by IR radiation.

In another aspect, the invention relates to the use of the combination described above in the preparation of formulations and compositions useful in the prevention of damages caused to the skin by IR radiation.

### EXAMPLES

Examples of particular embodiments are described below, without imposing any limitation to the scope of the invention beyond those comprised in the claims set out below.

### Example 1

### Effect of the Combination of the Invention on Gene Expression Induced By IR-A in Human Dermal Fibroblasts

To assess whether the combination of an Artemia saline extract with an Orica sativa extract including glycerol, is able to prevent damages induced by infrared radiation to human dermal fibroblasts, such cells were preincubated during 24 hours and post-incubated during 24 hours in the presence or absence of the combination of the invention. The aim was the evaluation of expression of MMP-1 enzymes (matrix metalloproteinase-1) induced by IR.

### Cell Culture

Human dermal fibroblasts prepared from neonatal foreskin were cultured in EMEM (Eagle's minimal essential medium, provided by PAA Laboratories GmbH, Germany) supplemented with 5% FCS (fetal calf serum, provided by Invitrogen, Germany), 1% L-glutamine, 1% non-essential amino acids (provided by Invitrogen, Germany), 1% streptomycin/B amphoterin (provided by Invitrogen, Germany) in humidified atmosphere containing 5% CO₂ during 4 days until reaching confluence, as described in J Invest Dermatol 128: 2491-2497, 2008. Only fibroblasts with less than 12 cell passages were used, to avoid changes in their original phenotypes during subculture. The cells were kept in 6-well plates for culture and irradiation.

### Cell Viability/Toxicity of Compounds

The cytotoxicity of compounds was evaluated by colorimetric evaluation with MTT, according to J Immunol Methods 65: 55-63, 1983, previously described in J Biol Chem 278: 47498-47507, 2003.

MTT (3-(4,5-dimethyl-2-thiazolyl)-2,5-diphenyl-2H-tetrazolium)bromide becomes bright blue and may form an insoluble precipitate when reduced in a cell, either enzymatically or through direct reaction with NADH (nicotinamide adenine dinucleotide) or NADPH (nicotinamide adenine dinucleotide phosphate). That is, the loss of color indicates decrease of cellular metabolism and increased toxicity.

Human dermal fibroblasts were seeded in 96-well plates, with 15,000 cells/200 μl into each well. The following day, the cells were treated with the substance of interest for 16 h. After 16 h, the medium was changed, 25 μl of MTT (2 mg/mL of PBS, phosphate buffered saline) were added and the mixture was incubated during 3 h. Finally, the solutions were removed, formazan crystals were dissolved in 200 μl of Me₂SO and the absorption was measured using a microplate reader at 540 nm in an Infinite 200 monochromator, provided by Tecan Deutschland GmbH, Germany. The viability was calculated as a percentage of control cells. Such cells were exposed only to the culture medium, and considered as 100% (according to Carcinogenesis 23: 1111-1120, 2002).

### In Vitro Irradiation

Primary human dermal fibroblasts were exposed to a dose of 360 J/cm² of infrared A-radiation previously determined as optimum to induce gene expression without affecting viability of this type of cell (J Invest Dermatol 128: 2491-2497, 2008). In short, the medium was exchanged for PBS, the caps were removed and the cells were exposed to infrared A-radiation (IRA) using a Hydrosun 500H IRA equipment, provided by Hydrosun Medizintechnik GmbH, Germany. The IR-A device is filtered with water, equipped with a black filter and emits wavelengths between 760 and 1400 nm, generating an irradiance of 360 mW/cm² at a distance of 20 cm, measured by means of an irradiance meter equipment Hydrosun HBM1, provided by Hydrosun, Medizintechnik GmbH, Germany. The culture discs were placed in a cooled plate connected to a thermostatic bath, provided by Thermo Haake GmbH, Germany, to keep temperatures below 37° C. during irradiation. Control cells were maintained in a thermostatic plate at 37° C., under similar conditions, without irradiation.

### Application of Compounds

The cells were kept in starvation during 24 h before irradiation (0% FCS) and the combination of the invention to be assessed was added during 24 h. Before the starvation period, before irradiation and before harvesting, the cells were washed once with PBS. During irradiation, the combination of the invention was not present (PBS incubation). After irradiation, the combination of invention was added again to fresh medium containing 2% FCS and was kept until cell harvesting 24 h after the treatment with IR-A.

The components of the combination of the invention were:
GP4G® SP: aqueous extract of Artemia saline
Orsirtina® GL: Oriza sativa extract, glycerol and water.

The viability data were first obtained to choose an appropriate concentration range, in which cell viability is not yet jeopardized, but effects can be observed. The viability was tested for each concentration in 8 duplicates. Each stimulation condition was tested in triplicate for evaluation of gene expression. RNA isolation and PCR

Total RNA was carried out as in Exp Dermatol 17: 771-779, 2008. For isolation of total RNA, RNeasy Total RNA kits were used, provided by Qiagen, Germany. The RNA concentration was determined by photometric measurement at 260/280 nm, with a an Eppendorf biophotometer (Germany). In the system Superscript® III First-Strand synthesis, provided by Invitrogen, Germany, aliquots of total RNA (100 ng) were applied to cDNA synthesis, a system for reverse transcription with random primers provided by Invitrogen, Germany). For each gene, a specific pair of primers was designed with Primer Express® 2.0 software, provided by Applied Bio Systems, Germany, based on the cDNA sequence published as indicated in the table below. Three independent experiments were carried out with 3 determinations each, and their average value was calculated. The PCR reactions were carried out with an Option 1 equipment, provided by Mi Research, USA, using SYBR Green® PCR Master Mix reagents, provided by Applied Bio Systems, Germany. Each sample was analyzed in duplicate using an universal protocol during 46 cycles. In more detail: activation: hot start polymerase for 10 minutes at 94° C.; penetration: 20 seconds at 95° C.; ringing: 20 seconds at 55° C.; extension: 30 seconds at 72° C. For comparison of PCR expression by real-time PCR, of control cells and treated cells, the method 2 (delta delta (C(T)) was employed, as described in Methods 25: 402-408, 2006.

### Marker Primers for Real Time PCR:

18S rRNA—Reference: Biochem J 232: 725-733, 1985
MMP-1—Reference: J Biol Chem 261: 6600-6605, 1986

### Statistics

One-way analysis of variance by ranks was used as a non-parametric test for comparing the differences between averages; p values of less than 0.05 were considered statistically significant (SigmaPlot 11.0 software, from Systat Software Inc., a US company).

### Results

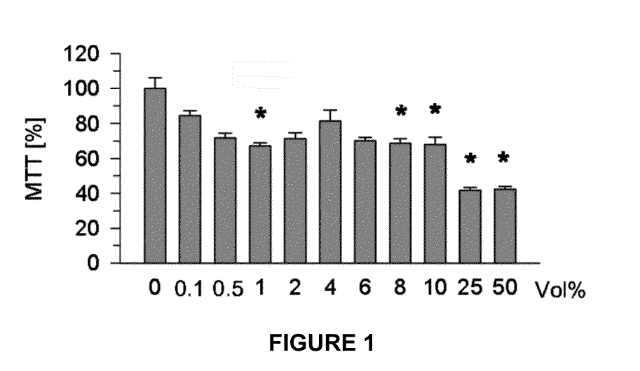

The effect of the combination of the invention on the viability of human dermal fibroblasts was evaluated, pursuant to FIG. 1. To determine an appropriate dosage range in which the viability of human dermal fibroblasts is not jeopardized during the 48 h incubation period, a wide range of percentages by volume (vol %), from 0.1% to 50%, was assessed. Since the inventive combination is soluble in water, solvent controls were not needed.

As can be seen in FIG. 1, concentrations below 6% don't affect cell viability during the 48 h incubation period. With such results, 3 concentrations below 6 vol % were chosen for the continuation of the test. To avoid non-effective low dosages, concentrations of 1% vol, 2% vol and 4% vol were used to test the tendency to inhibit over regulation of MMP-1 (matrix metalloproteinase-1=fibroblast collagenase) induced by IR-A in human dermal fibroblasts. The MMP-1 expression was determined by real-time PCR. The method 2 (delta delta C(T)) was used for comparing the relative gene expression with real-time PCR, control cells and treated cells. For this purpose, the value 1 was assigned to the expression of MMP-1/18S rRNA in untreated cells, and then, the corresponding ratios for other treatments were calculated.

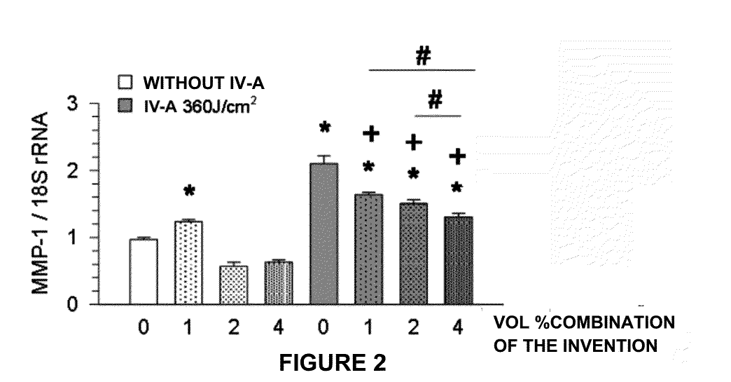

The average for non-treated dermal fibroblasts was set forth as 0.97±0.03. Increasing doses of the combination of the invention had little effect on the basal expression of MMP-1 (FIG. 2). A-infrared irradiation resulted in significant over regulation of the expression of MMP-1 of 2.1 times±0.12 SE (standard error). Increasing doses of the combination of the invention were able to significantly inhibit this expression of MMP-1 induced by IR-A—in detail: the combination of the invention reduced the expression of MMP-1 induced by IR-A 2.1 times to 1.3 time±0.03 SE (1 vol %), 1.5 time±0.06 SE (2 vol %) and 1.3 time±0.06 SE (4 vol %). All 3 concentrations of the assessed combinations of the invention significantly increased the expression of MMP-1 induced by IR-A in 45%, 55% and 73%. A 4 vol % dosage protected more than the 1 vol % or 2 vol % dosage from the over regulation of MMP-1 induced by IR-A, respectively.

As can be seen in FIG. 2, all doses assessed between 1 and 4 vol % of the combination of the invention partially inhibited the over regulation of MMP-1 induced by IR-A in human dermal fibroblasts. A dose-dependent effect was verified in the 45 to 73% range of inhibition.

TABLE 2
Inhibition of IR-A response in %

| Concentration of | | | |
|---|---|---|---|
| the combination | | | |
| of the invention | | | |
| (%) | +IR-A | % Response | % Inhibition |
| 0 | 2.1 ± 0.12 | 100 | 0 |
| 1 | 1.6^{+a} ± 0.03 | 55 | 45 |
| 2 | 1.5^{+b} ± 0.06 | 45 | 55 |
| 4 | 1.3^{+ab} ± 0.06 | 27 | 73 |

+significantly different from the isolated IR-A effect,
^{a},^{b}significantly different between both treatments (Kruskall-Wallis one-way analysis of variance by ranks)

### Example 2

### a Typical Sunscreen Formulation Containing the Combination of the Invention

| | | | CAS number |
|---|---|---|---|
| Chemical name - INCI/INN | % | Function | or source |
| BHT | 0.001 | ANTIOXIDANT | 128-37-0 |
| Butyl methoxydibenzoyl | 2.000 | SKIN | 70356-09-1 |
| methane | | PHOTOPROTECTOR | |
| 2-ethylhexyl 2-cyano-3,3- | 3.500 | SKIN | 6197-30-4 |
| diphenylacrylate | | PHOTOPROTECTOR | |
| (octocrylene) | | | |
| Glycerin | 2.00 | WETTING AGENT | 56-81-5 |
| Polysilicon-15 | 1.00 | SKIN | 207574-74-1 |
| | | PHOTOPROTECTOR | |
| Cetearyl alcohol | 1.80 | EMULSIFIER | 67762-27-0/ |
| | | | 8005-44-5 |
| C12-15 alkyl benzoate | 8.00 | EMOLLIENT | 68411-27-8 |
| Canola oil | 0.30 | EMOLLIENT/ANTIOXIDANT | 120962-03-0 |
| Diisopropyl adipate | 10.00 | EMOLLIENT | 6938-94-9 |
| Potassium cetyl phosphate | 2.80 | EMULSIFIER | 84861-79-0 |
| Vinylpyrrolidone/eicosene | 1.00 | FILM FORMER | 28211-18-9 |
| copolymer | | | |
| Titanium | 3.20 | SKIN | PARSOL ® TX, |
| dioxide/dimethicone/silica | | PHOTOPROTECTOR | (DSM |
| | | | Nutritional |
| | | | Products LLC, |
| | | | USA) |
| Dimethicone | 0.20 | EMOLLIENT | 63148-62-9/ |
| | | | 9006-65-9/00- |
| | | | 6-9016 |
| Xanthan gum | 0.05 | THICKENER | 11138-66-2 |
| Aminomethyl propanol | 1.65 | BUFFER | 124-68-5 |
| Di-sodium EDTA | 0.01 | SEQUESTRANT | 139-33-3 |
| Cyclopentasiloxane | 0.70 | EMOLLIENT | 541-02-6 |
| Caprylhydroxamic | 0.22 | WETTING AGENT | SPECTRASTAT ® |
| acid/caprylyl glycol/glycerine | | | (INOLEX |
| | | | CHEMICAL CO., |
| | | | USA) |
| Phenylbenzimidazole sulfonic | 3.00 | SKIN | 27503-81-7 |
| acid | | PHOTOPROTECTOR | |
| Silica | 0.01 | ABSORBENT | 7631-86-9/ |
| | | | 112945-52-5/ |
| | | | 60676-86-0 |
| Polyamide-5 | 1.45 | ABSORBENT | Orgasol ® Caresse |
| | | | (Lipo Chemicals, |
| | | | USA) |
| Ethylhexyl salicylate | 4.00 | SKIN | 118-60-5 |
| | | PHOTOPROTECTOR | |
| Orsirtine ® | 1.20 | SKIN | 7732-18-5/ |
| Water and Artemia extract | | CONDITIONER | 225234-40-2 |
| GP4G ® | 0.80 | SKIN | 7732-18-5/ |
| Water and extract of Oryza | | CONDITIONER | 90106-37-9 |
| sativa | | | |
| Tribehenin PEG-20 esters | 1.10 | EMULSIFIER | 220207-10-3 |
| Water | QSP | VEHICLE | 7732-18-5 |

### Manufacturing Mode:

### Phase 1 (Aqueous):

1. In the main reactor, the polymers of the aqueous phase are dispersed.
2. After complete dispersion on item 1, the other items of the aqueous phase are added, one by one, and mixed. Heating is initiated up to a temperature of 80-87° C.

### Phase 2 (Oily):

The components of the oily phase are added to the secondary reactor and melted at a temperature of 80-87° C.
Emulsion preparation: when both phases reached the temperature of 80-87° C., the oily phase was added over the aqueous phase and homogenize.
3. the emulsion was cooled to 35-40° C. and the other components of the formula were added. If necessary, pH was adjusted. the batch was completed with purified water, as necessary. Homegenization followed.

The information contained herein allows a person skilled in the art to carry out particular embodiments not expressly described, performing functions and achieving results of the same nature as those described, thus within the scope of protection of the claims attached.

1. A combination for skin protection against damages caused by infrared radiation comprising a SIRT1 peptide activator and a guanosine derivative alarmone.

2. The combination according to claim 1, wherein said SIRT1 peptide activator is selected from the group consisting of: fragments of proteins, peptide fragments, polypeptides and peptides, and any sequence of two or more amino acids linked by peptide bonds.

3. The combination according to claim 1, wherein said SIRT1 peptide activator is peptide fragments obtained from hydrolyzed rice extracts.

4. The combination according to claim 1, wherein said SIRT1 peptide activator is a G-L-Y-D-N-L-E Gly-Leu-Tyr-Asp-Asn-Leu-Glu peptide fragment.

5. The combination according to claim 1, wherein said guanosine derivative alarmone is selected from the group consisting of: guanosine monophosphate (RN: 85-32-5), guanosine diphosphate (RN: 146-91-8), guanosine triphosphate (RN: 86-01-1), 5′-diphosphate-3′-guanosine monophosphate (RN: 58902-76-4), guanosine tetraphosphate (RN: 33503-72-9), diguanosine tetraphosphate (RN: 4130-19-2), guanosine pentaphosphate (RN: 38918-96-6), and combinations thereof.

6. The combination according to claim 1 wherein said guanosine derivative alarmone is diguanosine tetraphosphate (RN: 4130-19-2).

7. The combination according to claim 1, wherein the content of the combination of SIRT1 peptide activator and guanosine derivative alarmone in relation to the total weight of the composition that contains it, varies between 0.01% and 2%.

8. The combination according to claim 1, wherein the ratio between SIRT1 peptide activator and guanosine derivative alarmone is in a range between 1:10 and 10:1.

9. The combination according to claim 1, wherein the SIRT1 peptide activator is contained in a product comprising water and glycerin and Oryza sativa extract; and the guanosine derivative alarmone is contained in an extract of Artemia sativa.

10. The combination, according to claim 8, wherein the ratios between SIRT1 peptide activator and guanosine derivative alarmone is in a range between 1:4 and 4:1.

11. The combination, according to claim 8, wherein the ratios between SIRT1 peptide activator and guanosine derivative alarmone is in a range between 1.5:1 and 1:1.5.

12. The combination, according to claim 1 wherein the combination is formulated in a form selected from the group consisting of: emulsion, cream, lotion, solution an anhydrous base, gel, paste, ointment, milk, liquid, aerosol, jelly and solid.

13. The combination, according to claim 12 wherein the anhydrous base is a lipstick or a powder.

14. The combination, according to claim 12 wherein the emulsion is formulated in a form selected from the group consisting of: oil-in-water, water-in-oil, silicone-in-water, water-in-silicone, water-in-oil-in-water and oil-in-water-in-oil emulsions.

15. A method for preventing and repairing of damages caused to the skin by infrared radiation comprising topically applying an effective amount of the combination of claim 1 onto the skin.


SEQUENCE LISTING (1 sequence)

SEQ ID NO: 1 - protein, 7 aa, ARTIFICIAL SEQUENCE
  SIRT1 GENE ACTIVATOR
GLYDNLE